(19) United States
(12) Patent Application Publication
(10) Pub. No.: US 2017/0119772 A1
(43) Pub. Date: May 4, 2017

(54) BEVERAGE COMPOSITION COMPRISING PHLOROTANNIN, GINSENG EXTRACT AND CAFFEINE

(71) Applicant: BOTAMEDI INC., Jeju-do (KR)

(72) Inventors: Haeng-Woo LEE, Bellevue, WA (US); Hyun-Cheol CHEON, Seoul (KR); Seong-Ho KIM, Daegeon (KR); Hyeon-Cheol SHIN, Daejeon (KR); Ju-Hee KIM, Jeju-do (KR); Jae-Keun OH, Seoul (KR)

(73) Assignee: BOTAMEDI INC.

(21) Appl. No.: 15/228,518

(22) Filed: Aug. 4, 2016

(30) Foreign Application Priority Data
Oct. 30, 2015 (KR) 10-2015-015186

(51) Int. Cl. A61K 31/522; A61K 36/258; A23L 2/52; A61K 47/26; A61K 47/12; A61K 47/02; A61K 31/357; A61K 9/00

(52) U.S. Cl. CPC A61K 31/522; A61K 31/357; A61K 36/258; A61K 9/0095; A61K 47/26; A61K 47/12; A61K 47/02; A23L 2/52; A23V 2002/00

(57) ABSTRACT

A beverage composition comprising phlorotannin, a ginseng extract and caffeine, and being effective in improving work performance, such as improving concentration and fatigue, preventing sleepiness, relieving hangovers, reducing headaches and improving physical abilities.

This application claims priority to and the benefits of Korean Patent Application No. 10-2015-0151868, filed with the Korean Intellectual Property Office on Oct. 30, 2015, the entire contents of which are incorporated herein by reference

### BACKGROUND OF THE INVENTION

1. Field of the Invention

The present disclosure relates to a beverage composition including phlorotannin, a ginseng extract and caffeine, and a drink including the beverage composition, and in particular, to a beverage composition effective in enhancing concentration, improving fatigue, preventing sleepiness, relieving hangovers, reducing headaches and improving physical abilities.

2. Description of the Related Art

As one method for enhancing work efficiency by concentrating longer on one's tasks in an ever-intensifying competitive society, demands for drinks based on the wakefulness effect of caffeine such as coffee or caffeinated drinks have increased.

Coffee has long been popularly consumed globally. Together with the recent increased consumption of caffeine-added energy drinks approximately 24% to 56% of young and middle-aged population drink coffee or energy drinks.

Generally, taking 400 mg or more of caffeine a day is known to cause sleeplessness, excessive nervousness, restlessness, hypersensitivity, nausea, stomach disorders, high blood pressure, increased pulses, muscle cramps and the like. In addition, excessive caffeine taking may increase the risks of lowered bone density and stomach cancer in the long run. Particularly, mixed intake of caffeine and alcohol is known to sharply increase cardiovascular risks.

While it becomes common to drink one to three cups of coffee containing high caffeine content, more serious problems occur as caffeine-added energy drinks also become common.

The amount of daily caffeine intake easily predicted from contemporary lifestyles may be summarized as follows. The amount of caffeine included in one cup (8 oz to 20 oz) of coffee in a coffee shop is approximately from 155 mg to 410 mg. In addition, the amount of caffeine included in one can (2 oz to 16 oz) of representative energy drink in the US is approximately from 80 mg to 214 mg.

Accordingly, one ingests 310 mg to 820 mg of caffeine when drinking two cups of coffee a day, and 465 mg to 1230 mg of caffeine when drinking three cups of coffee a day. Since many people often drink caffeinated energy drinks in addition to coffee, one may likely ingest excessive caffeine reaching 2 to 3 times of 400 mg, a daily caffeine limit.

Despite the fact that caffeine itself is classified as an active pharmaceutical ingredient, coffee or energy drinks are classified as a food which is under no particular regulation. Consequently, various side effects have occurred as cases of excessive caffeine intake (e.g., exceeding two to three cups of coffee or/and one to two cans of energy drinks a day) increase. Recently, many mortality cases have been reported that were caused by cardiovascular side effects such as an elevated blood pressure due to an excessive intake of energy drinks and an arrhythmia due to a mixed intake of caffeine and alcohol, and the like.

Despite such problems, the popularity fo the high caffeine drinks is thought to originate from a desire for concentrating for a longer period of time in various tasks and activities of modern days. Accordingly, it is much demanded to develop new drinks that is more helpful in performing mental and physical activities with reduced side effects from caffeine

### REFERENCES

### Non-Patent Documents

Burke et al, Effects of caffeine on the human circadian clock in vivo and in vitro. Science Translational Medicine. 2015, Sep. 16, Vol 7. Issue 305

Higgins J P, Tuttle T D, Higgins C L. Energy Beverages: Content and Safety. Mayo Clin Proc 85(11):1033-1041 (2010)

### SUMMARY OF THE INVENTION

The present disclosure is directed to providing, as a drink containing caffeine, a drink reducing side effects caused by caffeine by lowering the caffeine content in the drink, while capable of improving mental and physical performance.

In view of the above, an aspect of the present disclosure provides a beverage composition including caffeine, phlorotannin and a ginseng extract.

Another aspect of the present disclosure provides a drink including the beverage composition.

### DESCRIPTION OF THE PREFERRED EMBODIMENTS

The inventors of the present disclosure have found out that a drink including phlorotannin and a ginseng extract together with caffeine is capable of significantly improving mental and physical performance at lower caffeine contents compared to common caffeinated drinks, and have completed the present disclosure.

Accordingly, the present disclosure provides a beverage composition including caffeine, phlorotannin and a ginseng extract.

The present disclosure provides a drink including the beverage composition.

A proper amount of caffeine helps to enhance memory and concentration, to promote vitality and the like through wakefulness effects, however, side effects follow when it is taken excessively.

The drink of the present disclosure includes phlorotannin and a ginseng extract together with caffeine, and therefore, is capable of lowering the caffeine content. Consequently, caffeine side effects such as sleeplessness, excessive nervousness, restlessness, hypersensitivity, nausea, stomach disorders, increase blood pressure and pulse, and muscle cramps may be prevented.

In addition, the drink of the present disclosure has superior effects such as enhancing concentration, improving fatigue, preventing drowsiness, relieving hangovers, reducing headaches and improving physical abilities.

Hereinafter, substances of the beverage composition of the present disclosure will be described in more detail.

Phlorotannin is one type of polyphenol, is extracted from brown algae, and may be preferably obtained from one or more types selected from the group consisting of Ecklonia cava, Eisenia bicyclis, Ecklonia kurome, Ecklonia stolonifera, Ecklonia maxima, Ecklonia radiata and Eisenia arborea.

In addition, the phlorotannin may more preferably include one or more types of compounds selected from the group consisting of compounds of the following Chemical Formulae 1 to 10. The compounds of the following Chemical Formulae 1 to 10 commonly include a dibenzo-p-dioxin structure.

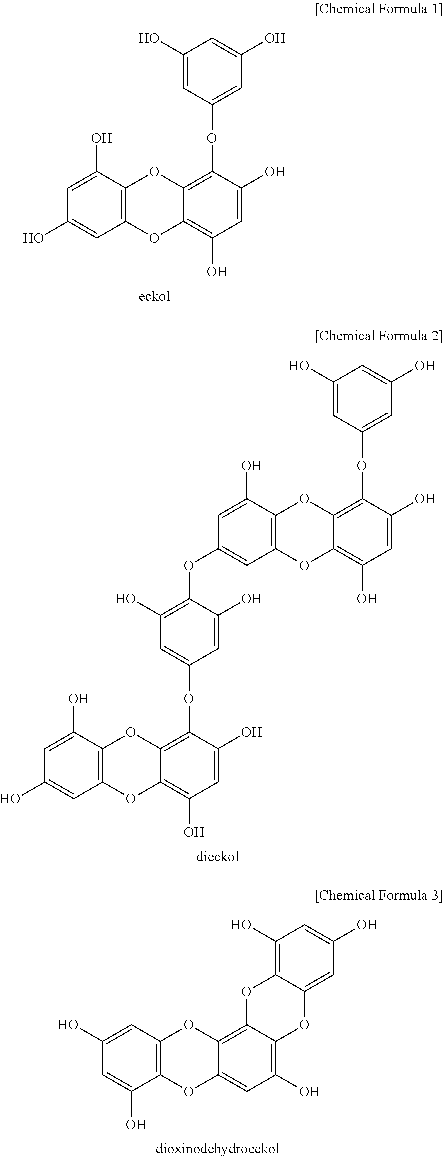 [SMILES: Oc1cc(O)cc(Oc2c(O)cc(O)c3c2Oc2c(O)cc(O)cc2O3)c1 ; Oc1cc(O)cc(Oc2c(O)cc(O)c3c2Oc2c(O)cc(Oc4c(O)cc(Oc5c(O)cc(O)c6c5Oc5c(O)cc(O)cc5O6)cc4O)cc2O3)c1 ; Oc1cc(O)c2oc3cc(O)c4oc5cc(O)cc(O)c5oc4c3oc2c1]

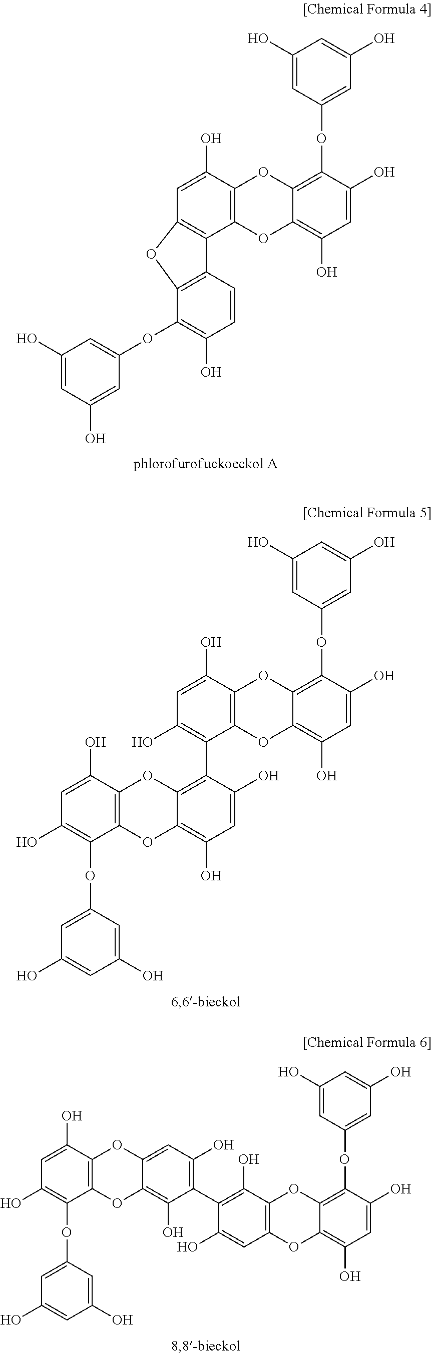 [SMILES: Oc1cc(O)cc(Oc2c(O)cc(O)c3c2Oc2c(O)cc4oc5c(Oc6cc(O)cc(O)c6)c(O)ccc5c4c2O3)c1 ; Oc1cc(O)cc(Oc2c(O)cc(O)c3c2Oc2c(O)cc(O)c(-c4c(O)cc(O)c5c4Oc4c(O)cc(O)c(Oc6cc(O)cc(O)c6)c4O5)c2O3)c1 ; Oc1cc(O)cc(Oc2c(O)cc(O)c3c2Oc2c(cc(O)c(-c4c(O)cc5c(c4O)Oc4c(Oc6cc(O)cc(O)c6)c(O)cc(O)c4O5)c2O)O3)c1]

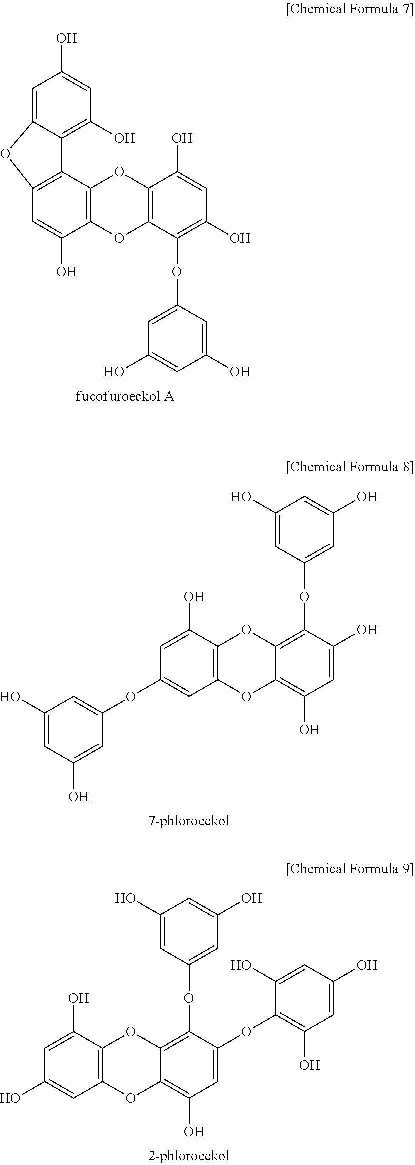 [SMILES: Oc1cc(O)cc(Oc2c(O)cc(O)c3c2Oc2c(O)cc4oc5cc(O)cc(O)c5c4c2O3)c1 ; Oc1cc(O)cc(Oc2cc(O)c3c(c2)Oc2c(O)cc(O)c(Oc4cc(O)cc(O)c4)c2O3)c1 ; Oc1cc(O)cc(Oc2c(Oc3c(O)cc(O)cc3O)cc(O)c3c2Oc2c(O)cc(O)cc2O3)c1]

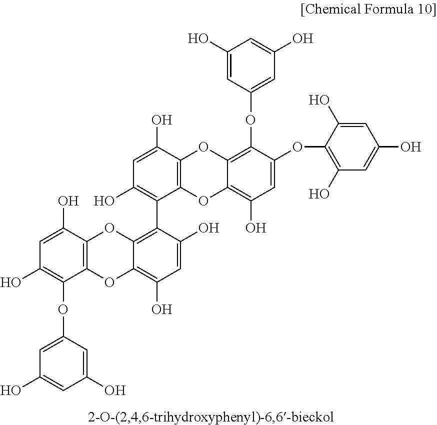 [SMILES: Oc1cc(O)cc(Oc2c(O)cc(O)c3c2Oc2c(O)cc(O)c(-c4c(O)cc(O)c5c4Oc4c(O)cc(Oc6c(O)cc(O)cc6O)c(Oc6cc(O)cc(O)c6)c4O5)c2O3)c1]

As for the content of the compounds of Chemical Formulae 1 to 10, the compound of Chemical Formula 1 may be included in 0.1% by weight to 6% by weight, the compound of Chemical Formula 2 in 5% by weight to 60% by weight, the compound of Chemical Formula 3 in 0% by weight to 30% by weight, the compound of Chemical Formula 4 in 0.5% by weight to 20% by weight, the compound of Chemical Formula 5 in 0.1% by weight to 10% by weight, the compound of Chemical Formula 6 in 0.5% by weight to 15% by weight, the compound of Chemical Formula 7 in 0% by weight to 5% by weight, the compound of Chemical Formula 8 in 0.1% by weight to 5% by weight, the compound of Chemical Formula 9 in 0.1% by weight to 10% by weight and the compound of Chemical Formula 10 in 0.1% by weight to 12% by weight, with respect to the total weight of the phlorotannin.

The ginseng extract of the present disclosure is prepared with red ginseng that underwent steaming and drying processes.

According to one embodiment of the present disclosure, red ginseng is introduced to an ethanol solution, an extract solvent, to obtain a first ginseng extract, and purified water is added to solids remaining after separating the first ginseng extract to obtain a second ginseng extract.

The first and the second ginseng extracts are mixed, filtered to remove precipitates, and then sterilized, and a ginseng extract may be obtained by filtering the sterilized ginseng extract once again, and vacuum concentrating the result.

The ginseng extract may include 3 mg/g to 22 mg/g of ginsenoside substances. The ginseng extract of the present disclosure may include one or more types selected from among Rg1, Rg2, Rb1 and Rg3 as the ginsenoside substance. More specifically, the ginseng extract may include 1 mg/g to 4 mg/g of the Rgl, 0 mg/g to 3 mg/g of the Rg2, 2 mg/g to 12 mg/g of the Rb1 and 0.1 mg/g to 2.5 mg/g of the Rg3.

By including the ginsenoside in the above-mentioned content, a mental and physical performance may be more improved.

The beverage composition of the present disclosure includes caffeine, and the caffeine may have a form of synthetic caffeine, or one or more types of natural caffeine extracts selected from the group consisting of guarana extracts, coffee extracts and tea extracts.

The beverage composition of the present disclosure uses a composition comprising caffeine and a mixture of a ginseng extract and phlorotannin in a weight ratio of 2:1 to 1:25, preferably in a weight ratio of 1:1 to 1:11, and more preferably in a weight ratio of 1:2 to 1:5.

When the caffeine, and the mixture of the ginseng extract and the phlorotannin are mixed in the above-mentioned weight ratio, mental and physical performance may be more enhanced while reducing the side effects of the caffeine at the same time.

In addition, the ginseng extract and the phlorotannin may be mixed in a weight ratio of 1:15 to 15:1, preferably in a weight ratio of 1:10 to 10:1, and more preferably in a weight ratio of 8:1 to 1:8.

When the ginseng extract and the phlorotannin are mixed in the above-mentioned weight ratio, mental and physical performances may be more enhanced.

The present disclosure also relates to a drink including the beverage composition of the the present disclosure.

The content of the beverage composition included in the drink of the present disclosure is from 0.05 wt % to 10 wt %, preferably from 0.1 wt % to 5 wt % and more preferably from 0.2 wt % to 3 wt %.

The present disclosure may provide a drink not exceeding daily caffeine intake of 400 mg by controlling the ratio of caffeine and a sum of a ginseng extract and phlorotannin (caffeine:red ginseng extract+phlorotannin) in the beverage composition included in the drink. The present disclosure may provide a drink that may be consumed 1 to 5 times a day within a daily caffeine intake limit.

In addition, the drink of the present disclosure may additionally include decaffeinated coffee in order to add taste and flavor, and may include one or more types selected from the group consisting of high fructose corn syrup, crystalline fructose, sucralose, citric acid, vitamin C, refined salts, potassium chloride and magnesium chloride.

The drink is effective in improving metal and physical performances, and more specifically is effective in enhancing concentration, reducing fatigue, preventing drowsiness, relieving hangovers, reducing headaches and improving physical abilities, and therefore, may be ingested as energy drinks.

Hereinafter, preferred examples and test examples are provided in order to illuminate the present disclosure. However, the following examples are provided for more readily understanding the present disclosure, and the scope of the present disclosure is not limited to the examples.

### PREPARATION EXAMPLE 1

### Extraction of Phlorotannin

Ecklonia cava was washed with water to remove foreign substances, dried in a shade, and then crushed.

500 g of the Ecklonia cava was placed in an extraction container, 20 times of a 30% by weight aqueous ethanol solution with respect to the weight of the Ecklonia cava was added thereto, and the result was reflux extracted for 4 hours at 75° C. to 80° C. to obtain an extract. The extract was filtered in a 10 μm filter element, and the filtered extract was vacuum concentrated at 55° C. to obtain a first extraction concentrate (solid content 55 wt %).

The first extraction concentrate was dissolved in warm water having a temperature of 50° C., then adsorbed to an adsorption resin, washed with water, and eluted with an ethanol solution to obtain a phlorotannin eluate. The eluate was filtered in a 1 μm filter element, and the filtered extract was vacuum concentrated at 55° C. to obtain a second extraction concentrate.

The second extraction concentrate was dried for 12 hours at 85° C. under a pressure of 70 torr using a vacuum drier, and then pulverized to obtain 50 g of phlorotannin extract powder.

When measuring the phlorotannin content in the phlorotannin extract powder using a Folin-Ciacaulteu method, the content was 97.5%, and the phlorotannin extract obtained above was filtered with a 0.2 μm membrane filter and loaded into high performance liquid chromatography.

In the high performance liquid chromatography, HP ODS Hypersil was used as a column, distilled water and methanol were used as a solvent, and the solvent was supplied at a flow speed of 1.0 ml/minute applying a linear gradient over 30 minutes from 15% to 70% of methanol to separate a total of 10 active materials. The active materials were identified as the compounds of Chemical Formulae 1 to 10.

In other words, it was identified that the phlorotannin extracted above included all the compounds of Chemical Formulae 1 to 10.

### PREPARATION EXAMPLE 2

### Preparation of Ginseng Extract

5 Year-old fresh ginseng was washed and arranged in a sieve plate, and then steamed for 3 hours at 97° C. After that, the result was first dried at a temperature of 60° C., second dried at a temperature of 50° C., and then sun dried to prepare original red ginseng.

400 g of the original red ginseng prepared above was introduced to an ethanol solution (70% ethanol by weight), an extractant, and a first ginseng extract was extracted for 36 hours at a temperature of 70° C.

Purified water was introduced to the solids remaining after separating the first ginseng extract, and a second ginseng extract was extracted for 24 hours at a temperature of 95° C.

The first and the second ginseng extracts were mixed, first filtered in a 100 μm filter element, then centrifuged for 20 minutes at 3,000 rpm using a rotary centrifuge to remove precipitates, and sterilized for 60 minutes in a constant-temperature water bath at 95° C.

The sterilized extract was second filtered in a 10 μm filter element, and the result was vacuum concentrated at 55° C. to prepare 280 g of a concentrated ginseng extract.

Red ginseng content in the ginseng extract was approximately 60% by weight.

The content of ginsenoside included in the ginseng extract obtained above was measured.

When measuring the content of ginsenoside Rg1, Rg2, Rb1 and Rg3 in the ginseng extract using a high performance liquid chromatography-ultraviolet detector (HPLC-UVD) method, the content of the ginsenoside Rg1, Rg2, Rb1 and Rg3 in the ginseng extract was 1.65 mg/g, 0.54 mg/g, 7.12 mg/g and 0.24 mg/g, respectively.

### TEST EXAMPLE 1

### Test on Improvement of Mental Work Performance

Using the phlorotannin prepared in Preparation Example 1, the ginseng extract prepared in Preparation Example 2 and synthetic caffeine, Composition 1 to Composition 14 were prepared according to the ratio listed in the following Table 1 and Table 2.

237 mL of decaffeinated coffee was placed in each of 14 cups having the same shape, size and material, and 240 mg of each of the Compositions 1 to 14, and 10 mg of red ginseng flavor were added thereto to prepare drinks of Comparative Examples 1 to 6 and Examples 1 to 8. The red ginseng flavor was added to minimize flavor differences between the drinks so that distinguishing the drinks is practically impossible.

In order to compare the effects of the drinks on the improvement of mental work performance, a panel of 10 office workers who drink at least two cups of coffee a day (excluding decaffeinated coffee drinker) was selected, and they were asked to drink one type of drink daily (different drinks between days), and a survey was carried out as follows before bedtime. They were asked to drink one cup at a time, and twice a day (9 am and 2 pm). During the test period, caffeine containing drinks such as caffeinated coffee and tea, and energy drinks were not allowed other than the given drinks.

<Survey>

1. During the time from the drink intake until bedtime, answer for the following items (1) to (3) with a score 0 to 4 compared to usual days.

(1) Degree of improvement in fatigue during work time.

(2) Degree of improvement in concentration during work time.

(3) Degree of improvement in overall work productivity.

<Evaluation Criteria>

0: There was no difference.

1: Slightly improved.

2: Somewhat improved.

3: Much improved.

4: Very much improved.

The results are shown in Table 1 and Table 2.

TABLE 1

| | Composition | Composition | Composition | Composition | Composition | Composition | Composition |
|---|---|---|---|---|---|---|---|
| Unit: mg | 1 | 2 | 3 | 4 | 5 | 6 | 7 |
| Caffeine | 240 | — | — | 120 | 120 | — | 80 |
| Red | — | 240 | — | 120 | — | 120 | 80 |
| Ginseng | | | | | | | |
| Extract | | | | | | | |
| Phlorotannin | — | — | 240 | — | 120 | 120 | 80 |
| | Comparative | Comparative | Comparative | Comparative | Comparative | Comparative | |
| | Example 1 | Example 2 | Example 3 | Example 4 | Example 5 | Example 6 | Example 1 |
| Survey (1) | 0.10 ± 0.32 | 0.60 ± 0.70 | 0.80 ± 0.63 | 1.00 ± 0.82 | 0.90 ± 0.57 | 1.10 ± 0.74 | 3.20 ± 0.63 |
| Survey (2) | 0.20 ± 0.42 | 0.10 ± 0.32 | 0.80 ± 0.42 | 1.10 ± 0.74 | 1.40 ± 0.52 | 1.60 ± 0.52 | 3.20 ± 0.92 |
| Survey (3) | 0.10 ± 0.32 | 0.20 ± 0.42 | 1.30 ± 0.67 | 1.10 ± 0.32 | 1.90 ± 0.74 | 1.70 ± 1.06 | 3.70 ± 0.48 |
| Total | 0.40 ± 0.70 | 0.90 ± 0.99 | 2.90 ± 1.20 | 3.20 ± 1.14 | 4.20 ± 1.23 | 4.40 ± 1.84 | 10.10 ± 1.52 |

TABLE 2

| | Composition | Composition | Composition | Composition | Composition | Composition | Composition |
|---|---|---|---|---|---|---|---|
| Unit: mg | 8 | 9 | 10 | 11 | 12 | 13 | 14 |
| Caffeine | 120 | 50 | 20 | 80 | 80 | 80 | 80 |
| Red | 60 | 95 | 110 | 110 | 140 | 50 | 20 |
| Ginseng | | | | | | | |
| Extract | | | | | | | |
| Phlorotannin | 60 | 95 | 110 | 50 | 20 | 110 | 140 |
| | Example 2 | Example 3 | Example 4 | Example 5 | Example 6 | Example 7 | Example 8 |
| Survey (1) | 2.80 ± 0.79 | 3.30 ± 0.48 | 2.50 ± 0.85 | 3.60 ± 0.52 | 2.80 ± 0.79 | 3.10 ± 0.88 | 2.60 ± 0.70 |
| Survey (2) | 3.50 ± 0.53 | 3.00 ± 0.82 | 2.10 ± 1.29 | 3.50 ± 0.53 | 2.90 ± 0.74 | 3.20 ± 0.63 | 2.80 ± 0.63 |
| Survey (3) | 3.20 ± 0.63 | 3.50 ± 0.71 | 2.80 ± 0.79 | 3.80 ± 0.42 | 3.10 ± 0.57 | 3.30 ± 0.67 | 2.80 ± 0.63 |
| Total | 9.50 ± 1.51 | 9.80 ± 1.69 | 7.40 ± 2.32 | 10.90 ± 0.88 | 8.80 ± 1.62 | 9.60 ± 1.35 | 8.20 ± 1.23 |

Compared to the drinks of Comparative Example 1 through Comparative Example 6 including caffeine, red ginseng extract or phlorotannin each alone, or including two substances thereof, it was seen that the drinks of Examples 1 to 8 including all three substances exhibited significantly superior results in each test item and the total. In other words, it was seen that, by combining the red ginseng extract and the phlorotannin together with the caffeine, superior synergistic effects were obtained in improving the work performance. In addition, it was seen that much superior effects were obtained even with substantially decreased caffeine content to ⅓.

### TEST EXAMPLE 2

### Evaluation of Smaller Size Drinks Convenient to Carry

A drink (50 mL) of Example 9 was prepared by adding various additives to Composition 11 that exhibited excellent effects in Test Example 1, and a drink (50 mL) of Example 10 was prepared using a guarana extract containing 12% of natural caffeine instead of synthetic caffeine.

A drink (50 mL) of Comparative Example 7 including caffeine, taurine and glucuronolactone was prepared. Also, 10 mL size drinks were prepared by reducing the amount of purified water from the compositions of Example 9 and Comparative Example 7 (Example 11 and Comparative Example 8). The compositions are shown in the following Table 3.

The additives added to the drinks of the tests were used to minimize distinction in tastes and flavors between drinks.

TABLE 3

| | Composition | Composition | | Composition | |
|---|---|---|---|---|---|
| Substance (mg) | 11 | 15 | Composition 1 | 11 | Composition 1 |
| Caffeine | 80 | 0 | 240 | 80 | 240 |
| Red Ginseng | 110 | 110 | 0 | 110 | 0 |
| Extract | | | | | |
| Phlorotannin | 50 | 50 | 0 | 50 | 0 |
| | | Example | Comparative | | Comparative |
| | Example 9 | 10 | Example 7 | Example 11 | Example 8 |
| Guarana Extract | 0 | 667 | 0 | 0 | 0 |
| Powder | | | | | |
| (Caffeine 12%) | | | | | |
| Taurine | 0 | 0 | 200 | 0 | 200 |
| Glucuronolactone | 0 | 0 | 120 | 0 | 120 |
| Red Ginseng | 10 | 10 | 10 | 10 | 10 |
| Flavor | | | | | |
| High Fructose | 2,000 | 2,000 | 2,000 | 2,000 | 2,000 |
| Corn Syrup | | | | | |
| Crystalline | 3,000 | 3,000 | 3,000 | 3,000 | 3,000 |
| Fructose | | | | | |
| Sucralose | 1 | 1 | 1 | 1 | 1 |
| Anhydrous Citric | 80 | 80 | 80 | 80 | 80 |
| Acid | | | | | |
| Refined Salts | 20 | 20 | 20 | 20 | 20 |
| Vitamin C | 30 | 30 | 30 | 30 | 30 |
| Potassium | 10 | 10 | 10 | 10 | 10 |
| Chloride | | | | | |
| Magnesium | 10 | 10 | 10 | 10 | 10 |
| Chloride | | | | | |
| Subtotal | 5,401 | 5,988 | 5,721 | 5,401 | 5,721 |
| Purified Water | 44,599 | 44,012 | 44,279 | 4,599 | 4,279 |
| Total Amount | 50,000 | 50,000 | 50,000 | 10,000 | 10,000 |

1. Sleepiness Preventing Effect

Tests were carried out for male bus drivers (60 people, age of 30 to 62) and male and female high school students (60 people, ratio of male and female 30:30, age of 16 to 18) in order to evaluate the preventing effects of sleepiness during driving or studying after taking the drinks of Examples 9 and 10, and Comparative Example 7.

For the evaluation, each of the drinks of Examples 9 and 10, and Comparative Example 7 was randomly provided to the participants after lunch between 1 pm and 2 pm and were asked to drink up the provided drink within one minute. At the same night, the participants were asked to evaluate the preventing effects of the drink on sleepiness during work hours with a score of 0 to 4. After the drink intake, taking caffeine drinks other than the drink was not allowed until the evaluation is finished.

Evaluation criteria were as follows, and the evaluation results are shown in the following Table 4.

<Evaluation Criteria>

0: Did not help.

1: A little helped.

2: Fairly helped.

3: Much helped.

4: Very much helped.

2. Hangover-Relieving Effects

Hangover-relieving effect was tested for the male and female office workers (48 people, ratio of male and female 36:12, age of 25 to 58) who had experienced difficulties in the work due to hangovers at least once a month. For the evaluation, the participants were asked to randomly take the drinks of Examples 9 and 10, and Comparative Example 7 in the order of participation between 7 pm and 11 pm, and asked to consume 1.5 times of each person's alcohol capacity that usually causes hangovers.

Based on the experiences until dinner time of the following day, the participants were asked to evaluate the hangover-relieving effects of the drink with a score of 0 to 4 compared to usual times without the drink.

Evaluation criteria were the same as the used for the effect of preventing sleepiness, and the evaluation results are shown in the following Table 4.

TABLE 4

| | | | Comparative |
|---|---|---|---|
| | Example 9 | Example 10 | Example 7 |
| Preventing Sleepiness | 3.20 ± 0.83 | 3.10 ± 0.79 | 2.10 ± 0.72 |
| (Bus Drivers) | | | |
| Preventing Sleepiness | 3.00 ± 0.86 | 3.15 ± 0.81 | 1.85 ± 0.88 |
| (High School Students) | | | |
| Relieving Hangovers | 2.81 ± 0.91 | 3.19 ± 0.75 | 0.44 ± 0.51 |
| (Office Workers) | | | |

According to the results shown in Table 4, the sleepiness-preventing effect increased by approximately 40% to 60%, and the hangover-relieving effect increased by approximately 6 times to 9 times in the drinks of Examples 9 and 10 compared to the drink of Comparative Example 7. While the hangover-relieving effect was not significantly shown in common caffeinated drinks, the drink of the present invention can greatly contribute to the work performance of office workers on the following day of overdrinking.

3. Headache-Relieving Effect

Using the drinks of Example 11 and Comparative Example 8, a headache-relieving effect was evaluated.

A headache-relieving effects of the drinks were tested for female office workers (20 people, age of 21 to 50) experiencing headaches at least twice a month using a crossover design.

Each participant was asked to take one randomly assigned drink a day for 4 weeks, and after the 4 weeks, was asked to evaluate the headache-relieving effect with a score of 0 to 4 compared to usual times.

The participants were asked to take a different drink daily for 4 weeks after 4 weeks of period with neither of the drinks of Example 11 or Comparative Example 8, and then were asked to evaluate the headache-relieving effect with a score of 0 to 4 compared to usual times.

Evaluation criteria were the same as the evaluation criteria used for the effect of preventing sleepiness, and the evaluation results are shown in the following Table 5.

4. Effect on Physical Endurance

Using the drinks of Example 11 and Comparative Example 8, an effect on physical endurance was evaluated.

Tests were carried out using a crossover design in male college students (20 people, age of 18 to 25) who are not professional athletes but do intensive exercise over one hour a day at least 3 times a week by evaluating how much the drinks were helpful in physical endurance.

Each participant was asked to take one randomly assigned drink a day for 4 weeks, and after the 4 weeks, was asked to evaluate how much the drink was helpful in physical endurance (when doing intensive exercise for 1 hour) with a score of 0 to 4 compared to usual times.

The participants were asked to take a different drink daily for 4 weeks after 4 weeks of period with neither of the drinks of Example 11 or Comparative Example 8, and then were asked to evaluate how much the drink was helpful in physical endurance with a score of 0 to 4 compared to usual times.

Evaluation criteria were the same as those used for the effect of preventing sleepiness, and the evaluation results are shown in the following Table 5.

TABLE 5

| | Example 11 | Comparative Example 8 |
|---|---|---|
| Effect of Reducing Headaches | 3.10 ± 0.88 | 0.30 ± 0.48 |
| (Female Office Workers) | | |
| Effect of Improving Exercise | 2.60 ± 1.17 | 1.60 ± 1.17 |
| Abilities (College Cyclists) | | |

From the results of Table 5, the drink of Example 11 was approximately 10 times more effective in reducing headaches and 1.6 times more effective in increasing physical endurance than the drink of Comparative Example 8.

5. Effect on Senior Fitness

Using the drinks of Example 11 and Comparative Example 8, effects on improving physical fitness such as flexibility, agility, balance and physical endurance, which are important in the activities of elderly, were evaluated. 40 Females aged 60 or older were randomly divided into two groups, and asked to take the drinks of Example 11 and Comparative Example 8, and measurements for the following 4 items were carried out before taking the drinks, and after 30 minutes from the drink intake.

<Evaluation Item>
(1) Flexibility (sit and reach): measuring the distance of fingertips going past one's feet while sitting and bending forward the upper part of the body in the unit of centimeters
(2) Agility: measuring the time taken to stand up from lying position in the unit of seconds
(3) Balance: measuring a time maintaining a balance when standing on left foot with one's right eye open
(4) Muscle endurance: measuring the number of repeating sitting in a chair and standing up.

The measurement results are shown in the following Table 6. In the group taking the drink of Example 11, flexibility, agility, balance and muscle endurance were significantly enhanced in 30 minutes compared to the group taking the drink of Comparative Example 8.

TABLE 6

| | | | Comparative |
|---|---|---|---|
| | | Example 11 | Example 8 |
| Seat and Reach | Before Drinking | 14.20 ± 3.74 | 14.85 ± 4.23 |
| (cm) | After Drinking | 16.75 ± 2.51 | 16.20 ± 3.82 |
| | Improved Rate (%) | 18.0 | 9.1 |
| Lie Down and | Before Drinking | 3.35 ± 0.43 | 3.07 ± 0.27 |
| Rise (sec) | After Drinking | 3.00 ± 0.34 | 2.87 ± 0.17 |
| | Improved Rate (%) | 10.4 | 6.5 |
| Stand on One | Before Drinking | 36.95 ± 16.33 | 49.95 ± 11.50 |
| Foot While | After Drinking | 66.20 ± 21.17 | 50.45 ± 13.22 |
| Opening Right | Improved Rate (%) | 79.2 | 1.0 |
| Eye (sec) | | | |
| Sit in Chair and | Before Drinking | 14.40 ± 1.34 | 14.70 ± 1.21 |
| Stand up | After Drinking | 17.40 ± 1.97 | 16.50 ± 1.37 |
| (Number of | Improved Rate (%) | 20.8 | 12.2 |
| Times) | | | |

Through the results of Test Examples 1 and 2, it was seen that the beverage composition of the present disclosure is effective in enhancing mental work performance, preventing sleepiness, relieving hangover, reducing headaches and improving physical abilities and the like while minimizing caffeine content.

Accordingly, when a drink is prepared using the beverage composition of the present disclosure, the drink is expected to be widely used as energy drinks for improving mental work performance and physical abilities in various occupational groups such as office workers, students, athletes and housewives.

The drink of the present disclosure is capable of lowering caffeine content to approximately 8% to 50% of the common caffeinated drinks, and therefore, is capable of preventing side effects caused by the caffeine.

In addition, the drink of the present disclosure is effective in enhancing concentration, relieving fatigue, preventing sleepiness, relieving hangovers, reducing headaches and improving physical abilities and the like.

What is claimed is:

1. A beverage composition comprising:
phlorotannin;
a ginseng extract; and
caffeine.

2. The beverage composition of claim 1, wherein the phlorotannin is extracted from the group consisting of Ecklonia cava, Eisenia bicyclis, Ecklonia kurome, Ecklonia stolonifera, Ecklonia maxima, Ecklonia radiata, Eisenia arborea, and combinations thereof.

3. The beverage composition of claim 1, wherein the phlorotannin comprises one or more compounds of the following Chemical Formulae 1 to 10:

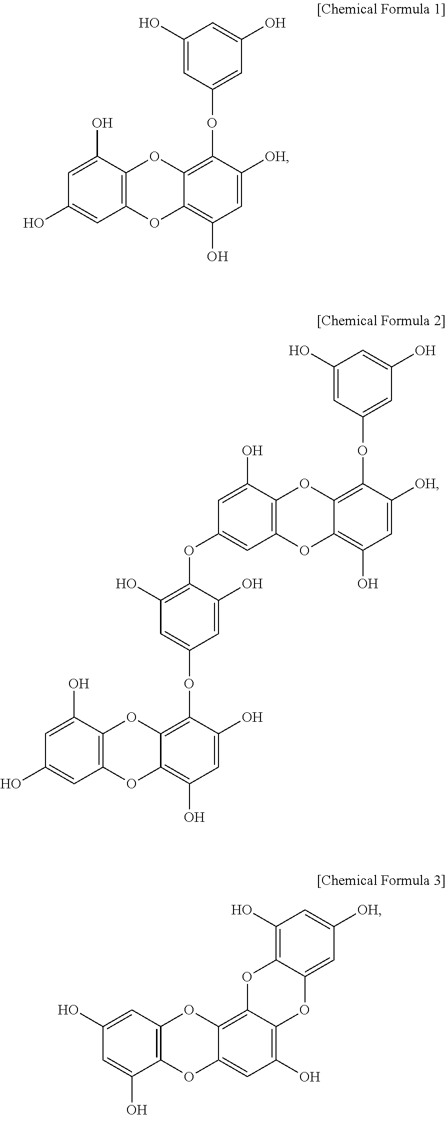 [SMILES: Oc1cc(O)cc(Oc2c(O)cc(O)c3c2Oc2c(O)cc(O)cc2O3)c1 ; Oc1cc(O)cc(Oc2c(O)cc(O)c3c2Oc2c(O)cc(Oc4c(O)cc(Oc5c(O)cc(O)c6c5Oc5c(O)cc(O)cc5O6)cc4O)cc2O3)c1 ; Oc1cc(O)c2oc3cc(O)c4oc5cc(O)cc(O)c5oc4c3oc2c1]

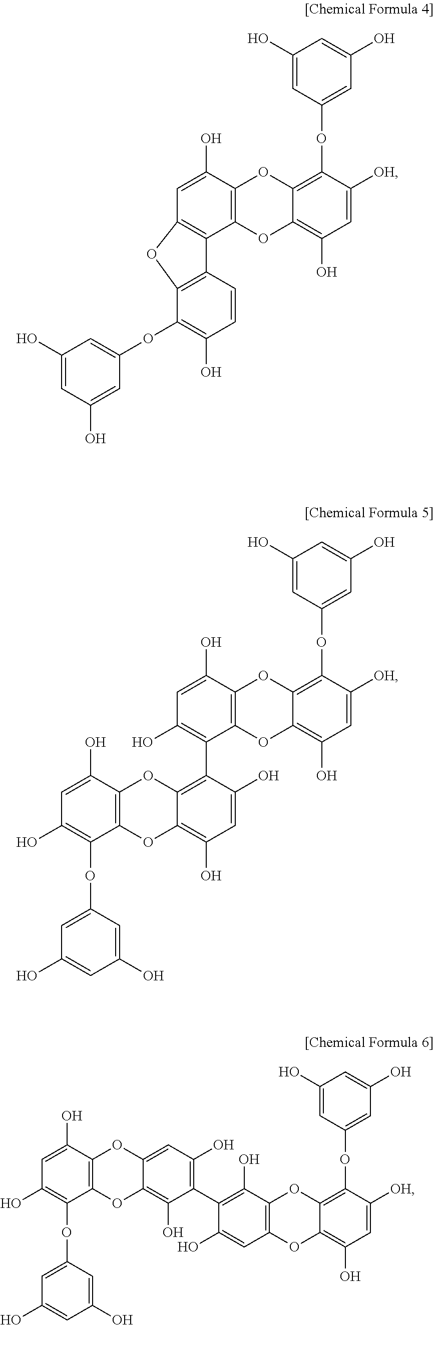 [SMILES: Oc1cc(O)cc(Oc2c(O)cc(O)c3c2Oc2c(O)cc4oc5c(Oc6cc(O)cc(O)c6)c(O)ccc5c4c2O3)c1 ; Oc1cc(O)cc(Oc2c(O)cc(O)c3c2Oc2c(O)cc(O)c(-c4c(O)cc(O)c5c4Oc4c(O)cc(O)c(Oc6cc(O)cc(O)c6)c4O5)c2O3)c1 ; Oc1cc(O)cc(Oc2c(O)cc(O)c3c2Oc2c(cc(O)c(-c4c(O)cc5c(c4O)Oc4c(Oc6cc(O)cc(O)c6)c(O)cc(O)c4O5)c2O)O3)c1]

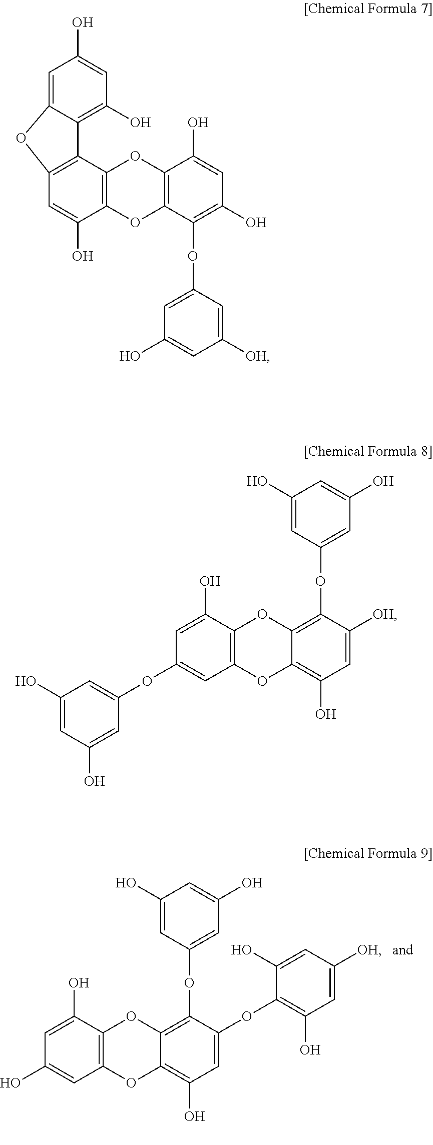 [SMILES: Oc1cc(O)cc(Oc2c(O)cc(O)c3c2Oc2c(O)cc4oc5cc(O)cc(O)c5c4c2O3)c1 ; Oc1cc(O)cc(Oc2cc(O)c3c(c2)Oc2c(O)cc(O)c(Oc4cc(O)cc(O)c4)c2O3)c1 ; Oc1cc(O)cc(Oc2c(Oc3c(O)cc(O)cc3O)cc(O)c3c2Oc2c(O)cc(O)cc2O3)c1]

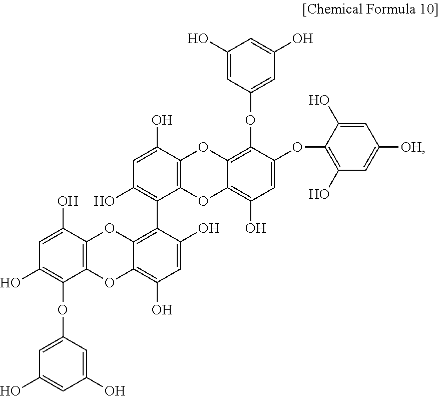 [SMILES: Oc1cc(O)cc(Oc2c(O)cc(O)c3c2Oc2c(O)cc(O)c(-c4c(O)cc(O)c5c4Oc4c(O)cc(Oc6c(O)cc(O)cc6O)c(Oc6cc(O)cc(O)c6)c4O5)c2O3)c1]

4. The beverage composition of claim 3, wherein, with respect to a total weight of the phlorotannin, the compound of Chemical Formula 1 is included in 0.1% by weight to 6% by weight, the compound of Chemical Formula 2 in 5% by weight to 60% by weight, the compound of Chemical Formula 3 in 0% by weight to 30% by weight, the compound of Chemical Formula 4 in 0.5% by weight to 20% by weight, the compound of Chemical Formula 5 in 0.1% by weight to 10% by weight, the compound of Chemical Formula 6 in 0.5% by weight to 15% by weight, the compound of Chemical Formula 7 in 0% by weight to 5% by weight, the compound of Chemical Formula 8 in 0.1% by weight to 5% by weight, the compound of Chemical Formula 9 in 0.1% by weight to 10% by weight and the compound of Chemical Formula 10 in 0.1% by weight to 12% by weight.

5. The beverage composition of claim 1, wherein the ginseng extract comprises 3 mg/g to 22 mg/g of ginsenoside.

6. The beverage composition of claim 5, wherein the ginsenoside comprises 1 mg/g to 4 mg/g of Rg 1, 0 mg/g to 3 mg/g of Rg2, 2 mg/g to 12 mg/g of Rb1 and 0.1 mg/g to 2.5 mg/g of Rg3.

7. The beverage composition of claim 1, wherein the caffeine is included in a form of synthetic caffeine, or one or more types of natural caffeine extracts selected from the group consisting of a guarana extract, a coffee extract and a tea extract.

8. The beverage composition of claim 1, wherein a mixing ratio of the caffeine, and a sum of the ginseng extract and the phlorotannin is from 2:1 to 1:25 in a weight ratio.

9. The beverage composition of claim 1, wherein a mixing ratio of the ginseng extract and the phlorotannin is from 1:15 to 15:1 in a weight ratio.

10. A drink comprising the beverage composition of claim 1.

11. The drink of claim 10 comprising 0.05 wt % to 10 wt % of the beverage composition.

12. The drink of claim 10, further comprising one or more types selected from the group consisting of high fructose corn syrup, crystalline fructose, sucralose, citric acid, vitamin C, refined salts, potassium chloride and magnesium chloride.
